(19) United States
(12) Patent Application Publication
(10) Pub. No.: US 2023/0346308 A1
(43) Pub. Date: Nov. 2, 2023

(54) METHOD OF MEASURING THE RESPONSE OF A PATIENT TO HYPOXIC TRAINING

(71) Applicant: Rainer Goytia, Berglen (DE)

(72) Inventor: Rainer Goytia, Berglen (DE)

(21) Appl. No.: 18/331,074

(22) Filed: Jun. 7, 2023

Related U.S. Application Data
(63) Continuation of application No. PCT/EP2021/083816, filed on Dec. 1, 2021.

(30) Foreign Application Priority Data
Dec. 11, 2020 (DE) 10 2020 215 742.1

(51) Int. Cl. A61B 5/145; G16H 50/30; A61B 5/00

(52) U.S. Cl. CPC A61B 5/4884; A61B 5/14542; G16H 50/30; A61M 16/0045

(57) ABSTRACT

A method of measuring a hypoxic session index as a measure of the response of a patient to hypoxic training with measurement of an index that characterizes the oxygen content in the patient’s blood is disclosed. The index is the oxygen saturation and/or the partial pressure of oxygen. The patient is first supplied with a normoxic gas mixture and then supplied with a hypoxic gas mixture. Subsequently, the patient receives a normoxic or hyperoxic gas mixture in a reoxygenation phase. The reoxygenation phase extends from the juncture from which the patient is supplied with the normoxic or hyperoxic gas mixture, over a defined hyperoxic period, wherein the index attains a hyperoxic reference value of the index, and over a subsequent predetermined concluding period. Differences between a data curve having the measurements of the index plotted against the respective measurement time and a predetermined reference curve ascertain the hypoxic session index.

### CROSS REFERENCE TO RELATED APPLICATIONS

This continuation application claims priority to PCT/EP2021/083816 filed on Dec. 1, 2021 which has published as WO 2022/122511 A1 and also the German application number 10 2020 215 742.1 filed on Dec. 11, 2020, the entire contents of which are fully incorporated herein with these references.

### DESCRIPTION

### Field of the Invention

The invention relates to a method of measuring a hypoxic session index as a measure of the response of a patient to hypoxic training or to a hypoxic session, with measurement of an index that characterizes the peripheral oxygen saturation in the patient’s blood and/or the response of the pulse. The invention further relates to a device for carrying out the method.

### BACKGROUND OF THE INVENTION

Methods for performing a hypoxic therapy session are known from the prior art:

DE 10 2012 010 806 A1 discloses a hypoxic therapy session in which the values of peripheral oxygen saturation and of the pulse of a patient are sent to a monitoring device, while a hypoxic gas mixture is supplied to the patient in alternation with a normoxic or hyperoxic gas mixture. The hypoxic gas mixture and the normoxic or hyperoxic gas mixture are supplied in each case over a predetermined hypoxic period.

The known methods provide comparatively little support in the evaluation of the data obtained in hypoxic training.

### SUMMARY OF THE INVENTION

### Object of the Invention

It is therefore an object of the present invention to specify a method in which the data obtained in hypoxic training are evaluated in the form of quantitative results. It is also an object to provide a device for carrying out the method.

### Brief Description of the Invention

This object is achieved according to the invention by a method according to claim 1 and a device according to claim 14. Advantageous embodiments result from the respectively dependent claims.

The method according to the invention includes the following steps:
I. Supplying the patient with a normoxic gas mixture in an initial phase over a defined first period;
II. Determining the mean value of the index in the initial phase;
III. Supplying the patient with a hypoxic gas mixture in a hypoxic phase over a defined hypoxic period;
IV. Supplying the patient with the normoxic or hyperoxic gas mixture in a reoxygenation phase over a defined hyperoxic period and in a subsequent predetermined concluding period;
V. Plotting the index against time in the initial phase, the hypoxic phase and the reoxygenation phase in the form of a data curve;
VI. Determining the hypoxic session index from a difference between the data curve and a predetermined reference curve.

The difference between the data curve and the reference curve allows for a quantitative evaluation of the measured values of oxygen saturation. The index is in particular the oxygen saturation or the partial pressure of oxygen dissolved in the blood of the patient, the relationship of which is represented by the oxygen binding curve having a sigmoidal profile. The difference is determined, in particular, by a distance between the data curve and the reference curve or by a comparison of surfaces that are limited by the data curve or the reference curve.

A hypoxic safety value, which, in particular, indicates the smallest value to which the index may drop in the hypoxic phase, is in particular between 70% and 88% of the mean value of the index in the initial phase, in particular 80% of the mean value of the index in the initial phase.

In the initial phase, the patient is, in particular, under no physical stress and breathes ambient air. The patient is in a resting position, in particular lying on a couch. In the initial phase, the device for performing the measurement of the hypoxic session index is calibrated, in particular. The initial phase is in particular between 1 minute and 5 minutes. In the initial phase, the index, for example the partial pressure of oxygen, is measured and recorded in each second, in particular in a period between 20 seconds and 120 seconds, in particular 30 seconds before the start of the first hypoxic phase. After the end of the initial phase, the mean value of the index is calculated for determining a baseline of the index during hypoxic training. The defined hypoxic period and/or hyperoxic period is in particular between 1 minute and 15 minutes. The mean value of the index in the initial phase according to step II is determined in particular before the hypoxic phase according to step III, preferably during the initial phase or at the end of the initial phase. In an alternative embodiment of the method, the hypoxic phase according to step III and the hyperoxic phase according to step IV are interchanged.

### Preferred Embodiments of the Invention

An advantageous embodiment of the method is characterized in that the reference curve is a data curve which is determined for a reference person after steps I to V, or a data curve which is calculated by averaging data curves of a plurality of reference persons, wherein the data curves are determined after steps I to V for the reference persons.

A reference person is, in particular, a person which shows measured values of the index that are classified as healthy. The reference curve can also be ascertained by averaging the measured values of the index of the reference persons at the respective same time of the method for several reference persons. If necessary, the reference persons can be grouped according to their physical characteristics.

In some embodiments of the method, the hypoxic session index is shown as a list of indices, in particular indices that are shown in the following text.

The method is advantageously characterized by setting a hypoxic cycle score (HCS) in the hypoxic phase for determining the hypoxic session index, the following steps being performed:
VII. Setting an HCS reference value of the index that is smaller than the mean value of the index in the initial phase and is greater than the hypoxic safety value;
VIII. Plotting the HCS reference value against time in the hypoxic phase as an HCS reference line of the hypoxic phase;
IX. Determining an HCS determination area as an area between the data curve and the HCS reference line in the hypoxic phase;
X. Determining an HCS reference area as an area between the reference curve and the HCS reference line in the hypoxic phase;
XI. Determining an HCS area difference between the HCS reference area and the HCS determination area and/or an HCS area ratio as the ratio of the HCS determination area and the HCS reference area;
XII. Determining the value of the hypoxic cycle score as a measure of the HCS area ratio and/or the HCS area difference.

The HCS serves to compare the drop in the reference curve and the data curve in the hypoxic phase.

The HCS reference value is, in particular, between 88% and 92% of the mean value of the index from the initial phase, preferably 90% of the mean value of the index from the initial phase.

The HCS determination area is defined, in particular, as an area between the data curve from the time at which the index on the data curve has dropped to the HCS reference value, up to the end of the hypoxic phase and the HCS reference line from the time at which the index has dropped to the HCS reference value, up to the end of the hypoxic phase.

The HCS reference area is defined, in particular, as an area between the data curve from the time at which the index on the reference line has dropped to the HCS reference value, up to the end of the hypoxic phase and the HCS reference line from the time at which the index has dropped to the HCS reference value up to the end of the hypoxic phase.

The hypoxic cycle score (HCS) is determined, in particular, by the absolute difference between the HCS determination area and the HCS reference area, which is defined in particular by the HCS area difference. Alternatively, the hypoxic cycle score is determined by the relative difference between the HCS determination area and the HCS reference area, in which the HCS area difference is related to the HCS reference area. The relative difference is given, in particular, in percent.

A preferred embodiment of the method is characterized by setting a reoxygenation max score (RMS) in the reoxygenation phase for determining the hypoxic session index by the following steps:
XIII. Setting an RMS safety time after the start of the reoxygenation phase;
XIV. Determining the RMS safety value of the index associated with the RMS safety time on the data curve in the reoxygenation phase;
XV. Plotting the RMS safety value against time in the reoxygenation phase as an RMS safety line of the reoxygenation phase;
XVI. Determining an RMS determination area as an area between the data curve and the RMS safety line in the reoxygenation phase;
XVII. Determining an RMS reference area as an area between the reference curve and the RMS safety line in the reoxygenation phase;
XVIII. Determining an RMS area difference between the RMS reference area and the RMS determination area and/or an RMS area ratio as the ratio of the RMS determination area and the RMS reference area;
XIX. Determining the reoxygenation max score as a measure of the RMS area ratio and/or the RMS area difference.

The reoxygenation max score serves to compare the reference curve and the data curve in the reoxygenation phase.

The RMS safety time is preferably 30 seconds to 50 seconds, preferably 45 seconds, after the start of the reoxygenation phase. The RMS safety time is preferably a time at which the index assumes between 3% and 10% of the mean value of the index from the initial phase.

The RMS determination area is defined, in particular, as an area between the data curve from the RMS safety time to the end of the reoxygenation phase and the RMS safety line from the RMS safety time to the end of the reoxygenation phase.

The RMS reference area is defined, in particular, as an area between the reference curve from the RMS safety time to the end of the reoxygenation phase and the RMS safety line from the RMS safety time to the end of the reoxygenation phase.

The reoxygenation max score (RMS) is determined, in particular, by the absolute difference between the RMS determination area and the RMS reference area, which is defined in particular by the RMS area difference. Alternatively, the reoxygenation max score is determined by the relative difference between the RMS determination area and the RMS reference area, in which the RMS area difference is related to the RMS reference area. The relative difference is given, in particular, in percent.

The reference curve increases, in particular, to 99% of the value of the index which the index can reach at most in the initial phase. If the index is the partial pressure of oxygen or the oxygen saturation, the reference curve will increase in particular to 99% of the partial pressure of oxygen or to 99% of the oxygen saturation as the most healthy value of the index; this applies to those cases where the entire hemoglobin in the blood is loaded with oxygen. The reference curve will preferably increase to the most healthy value of the index in a period from 10 seconds to 14 seconds, preferably 12 seconds, after the RMS safety time.

A development of the above-mentioned embodiment of the method is characterized by determining a user reoxygenation potential as a measure of the difference between a predetermined reference reoxygenation max score and the reoxygenation max score determined in step XIX. The user reoxygenation potential is, in particular, the aforementioned difference, expressed in percent. The reference reoxygenation max score is, in particular, 99% of the maximum value which the index can reach in the initial phase.

Preferred embodiments of the method are characterized by setting a dynamic score in the hypoxic phase for determining the hypoxic session index, having the steps: XX. Determining a DS reference time at which the index in the hypoxic phase has dropped to a defined DS reference value, wherein the DS reference value is smaller than the mean value of the index in the initial phase and greater than the hypoxic safety value; XXI. Determining the dynamic score as a measure of the time difference between the DS reference time and a defined DS reference time interval.

The dynamic score serves to indicate the response time of the body of a patient to oxygen deficiency, measured from the start of the hypoxic phase, in particular, until a response of the body can be measured at a sensor.

The defined DS reference value is, in particular, 95% to 98%, preferably 97% of the mean value of the index from the initial phase. The DS reference time interval is, in particular, between 40 seconds and 50 seconds, preferably 45 seconds. The time difference between the DS reference time and the DS reference time interval is recorded as the value of the dynamic score. In case of negative time difference values and/or time difference values that are greater than a predetermined value, in particular more than 184 seconds, the dynamic score is preferably set to zero. Alternatively or additionally, the measurement time in the initial phase and the hypoxic phase may be divided into time intervals, wherein each time interval is assigned a hypoxic improvement potential such that a position of the DS reference time in a certain time interval corresponds to a certain hypoxic improvement potential

The method is advantageously characterized by setting a reoxygenation impulse score in the reoxygenation phase for determining the hypoxic session index, having the steps:
XXII. Determining an RI reference time at which the index in the reoxygenation phase has increased to a defined hyperoxic reference value;
XXIII. Determining the reoxygenation impulse score as a measure of the time difference between the RI reference time and a defined RI reference time interval.

The reoxygenation impulse score serves to indicate the response time of the body of a patient to a hyperoxic gas mixture, measured from the start of the hyperoxic phase, in particular until a response of the body to the hyperoxic gas mixture can be measured by a sensor.

The defined hyperoxic reference value is, in particular, 2% to 5%, preferably 3% of the mean value of the index from the initial phase. The RI reference time interval is, in particular, between 40 seconds and 50 seconds, preferably 45 seconds.

In case of negative time difference values and/or time difference values that are greater than a predetermined value, in particular, more than 184 seconds, the reoxygenation impulse score is preferably set to zero. Alternatively or additionally, the measurement time in the reoxygenation phase may be divided into time intervals, wherein each time interval is assigned a reoxygenation improvement potential such that a position of the RI reference time in a certain time interval corresponds to a certain reoxygenation improvement potential.

One embodiment of the method is characterized by setting an oxygen recovery score in the reoxygenation phase for determining the hypoxic session index by the reoxygenation impulse score and the reoxygenation max score, in particular, by averaging.

Averaging is, in particular, an arithmetic averaging.

Advantageously, the index of the oxygen content is the partial pressure of oxygen and/or the oxygen saturation. These indices indicate what percentage of the total hemoglobin in the blood of a patient is loaded with oxygen.

A further embodiment of the method is characterized by determining a baseline potential as a measure of the difference between a predetermined ideal value of the oxygen saturation index and the mean value of the oxygen saturation index from the initial phase. The predetermined ideal value of the oxygen saturation index is, in particular, 99% of the value of the index which the index can reach at most in the initial phase.

An advantageous embodiment of the method is characterized by setting a lower boundary line and an upper boundary line, wherein the lower boundary line in the hypoxic phase shows smaller values of the index than the reference curve, and the upper boundary line shows greater values of the index than the reference curve at the respective same measurement times, wherein the boundary lines are determined, in particular, from measurements of the index in one or more subjects, wherein only values of the index are considered for determining the hypoxic session index, which values are smaller than the value of the index in the upper boundary line and greater than the value of the index in the lower boundary line at the time of measurement of the respective value of the index.

In particular, only data points which lie in the area defined by the reference curves are used to calculate the reference areas and determination areas mentioned in the application. In the context of the application, this area is referred to as the normal hypoxic range and defines the range of the valid measured values. Values of the index that are outside the area defined by the boundary line at the respective time are classified as incorrect measurement values, for example as a result of poor contact between the sensor and the patient. The boundary lines are, in particular, previously measured data curves of subjects with suitable values of the index for the upper and lower boundary line.

A preferred embodiment of the method is characterized by determining the hypoxic session index by changing a pulse curve of the patient during steps III to V. In particular, an increase or decrease in the pulse during hypoxic training may be used as an indicator of the response of the body to hypoxic training.

A development of the above-mentioned embodiment of the method is characterized by determining a heart rate relaxation score by plotting a pulse curve against time during hypoxic training, having the following steps:
XXIV. Determining the mean value of the pulse in the initial phase;
XXV. Plotting the mean of the pulse against the duration of the hypoxic training after the initial phase as a heart rate baseline parallel to the time axis, wherein the heart rate baseline forms a first leg of a relaxation measurement angle;
XXVI. Plotting a second leg of the relaxation measurement angle, wherein the second leg runs through the pulse at the start time of the hypoxic phase and through the point of the pulse curve having the lowest value of the pulse after the initial phase;
XXVII. Determining the heart rate relaxation score as a measure of the relaxation measurement angle.

The heart rate baseline is determined, in particular, in the initial phase, wherein the pulse is measured and recorded in every second in order to determine the heart rate baseline from the mean value of the pulse in the initial phase. The heart rate relaxation score is used to indicate the change in the pulse during hypoxic training. This indicates relaxation of the patient during hypoxic training.

The maximum value of the pulse above the heart rate baseline is used to determine a negative heart rate relaxation score, and the smallest value of the pulse below the heart rate baseline is used to determine a positive heart rate relaxation score. The positive heart rate relaxation score, in particular, is used to determine the response of the patient’s body to hypoxic training.

One embodiment of the method is characterized by one or more repetitions of steps III to V before determining the hypoxic session index according to step VI. In each repetition, referred to, in particular, as a cycle, one or more of the aforementioned indices can be determined in order to form mean values for the respective index therefrom or to compare values of the respective indices in order to detect values incorrectly measured in a cycle.

A device for carrying out a method according to one of the aforementioned embodiments comprises a mask for supplying the hypoxic, normoxic and/or hyperoxic gas mixture to the patient, a controller for controlling the device, and a finger clip for measuring the pulse and/or the index.

Such a device makes it possible to quantitatively detect suitable indices for determining the response of the body of a patient.

One embodiment of the device comprises a mobile application for representing the hypoxic session index and/or at least one of the indices for determining the hypoxic session index according to one of the aforementioned embodiments. The hypoxic session index can be represented by colors of a color scale in order to allow for rapid detection of the response of the body of a patient to hypoxic training. Alternative embodiments of the device comprise a stationary application for representing the hypoxic session index and/or at least one of the indices for determining the hypoxic session index according to one of the aforementioned embodiments.

Further advantages of the invention can be found in the description and the drawings. Likewise, the aforementioned features and those which are to be explained below can each be used individually or together in any desired combinations. The embodiments shown and described are not to be understood as an exhaustive list, but, rather, have an exemplary character for the description of the invention.

### BRIEF DESCRIPTION OF THE INVENTION AND DRAWINGS

FIG. 1 schematically shows a device for measuring the response of a patient to hypoxic training;

FIG. 2 schematically shows an overview of entries measured by the device, through which an indication value comprising a hypoxic session index and/or a heart rate relaxation score as a measure of the response of a patient to hypoxic training is determined;

FIG. 3 schematically shows a method of measuring the hypoxic session index HSI;

FIG. 4 schematically shows a data curve of an index which is recorded in the method;

FIG. 5 schematically shows the data curve up to a DS reference time at which the index has dropped to a defined DS reference value;

FIG. 6 schematically shows a lower boundary line and an upper boundary line of the index in a hypoxic phase;

FIG. 7 schematically shows the data curve from the beginning of a reoxygenation phase up to an RI reference time at which the index has increased to a hyperoxic reference value;

FIG. 8 schematically shows the reference curve as well as a first data curve and a second data curve in the reoxygenation phase above an RMS safety value;

FIG. 9 schematically shows a pulse of the patient plotted against the measurement time in the form of a pulse curve;

FIG. 10 schematically shows the device with a display.

### DETAILED DESCRIPTION OF THE PREFERRED EMBODIMENTS

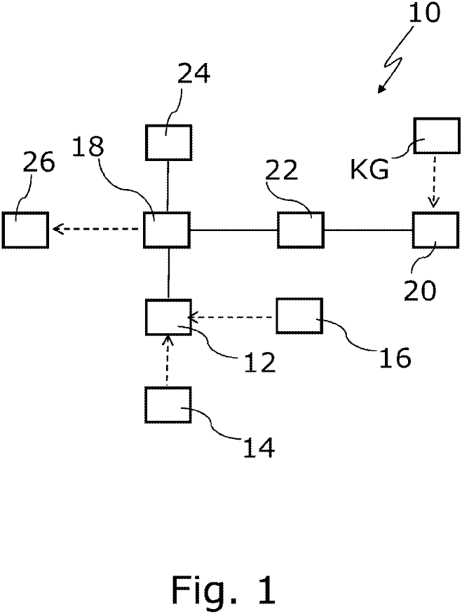

FIG. 1 schematically shows a device 10 for measuring the response of a patient to hypoxic training. The device comprises a mask 12 for supplying a hypoxic gas mixture 14, a normoxic and/or hyperoxic gas mixture 16 to a patient (not shown).

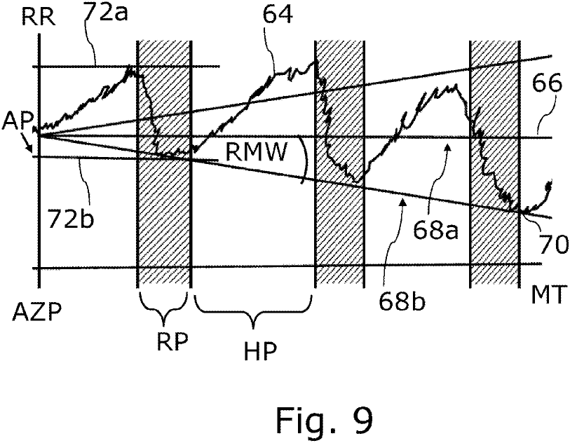

In addition, the device has a controller 18 for controlling the device 10 and a finger clip 20 and a sensor 22, in particular, arranged in the finger clip 20, for measuring a pulse RR (see FIG. 9) of the patient and/or an index KG which characterizes the oxygen saturation of the patient’s blood, for example of the partial pressure of oxygen. The device 10 comprises a mobile application 24 for representing an indication value 26 by which the response of the patient’s body to hypoxic training is quantitatively detected.

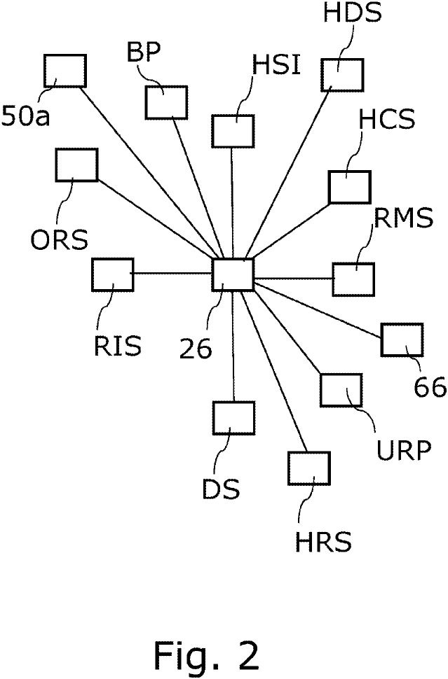

FIG. 2 schematically shows an overview of the entries by which the indication value 26 is determined. These entries comprise a hypoxic session index HSI, a hypoxic cycle score HCS, a reoxygenation max score RMS, a user reoxygenation potential URP, a dynamic score DS, a reoxygenation impulse score RIS, an oxygen recovery score ORS, a baseline potential BP, a hypoxic baseline 50a, a heart rate baseline 66, a heart rate relaxation score HRS and/or a heart rate dynamic score HDS. These entries or indices can be shown in summarized form, for example in the form of averaging, or separately, for example in list form. The hypoxic session index HSI results, in particular, from one or more of the other aforementioned indices, for example by averaging these indices.

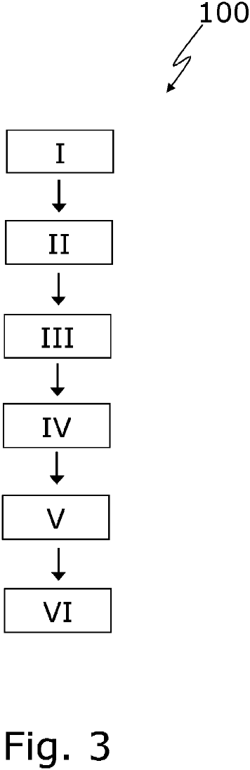

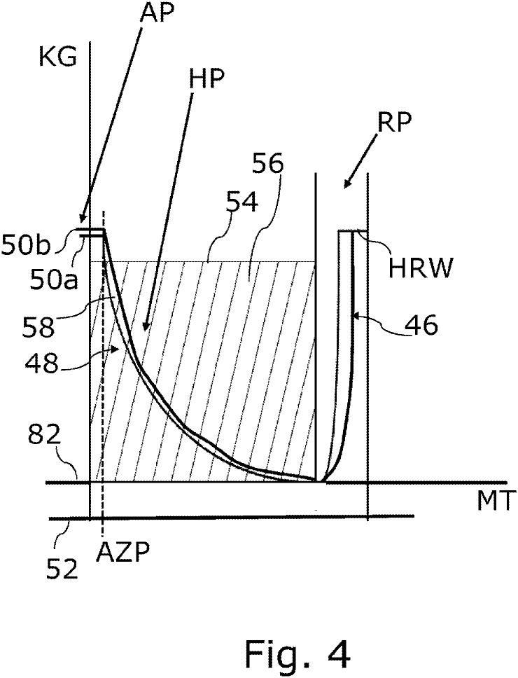

FIG. 3 schematically shows a method 100 of measuring the response of a patient to hypoxic training. In a first step I, the patient is supplied with the normoxic gas mixture 16 in an initial phase AP (see FIG. 4), in particular, in a rest position, over a defined first period, in particular over a first period of 20 seconds to 40 seconds, preferably 30 seconds. In a second step II, the mean value of the index KG in the initial phase AP is calculated. In a third step III, after the initial phase AP, the patient is supplied with a hypoxic gas mixture 14 over a defined hypoxic period in a hypoxic phase HP (see FIG. 4) until the index KG drops to a hypoxic value 82 which is greater than a defined hypoxic safety value 52 (see FIG. 4). In a fourth step IV, the patient (not shown) is supplied with a normoxic or hyperoxic gas mixture 16 over a defined hypoxic period in a reoxygenation phase RP (see FIG. 4), wherein the index KG attains a hyperoxic reference value HRW (see FIG. 4), and over a subsequent predetermined concluding period. In a fifth step V, the index KG is plotted against time in the initial phase AP, the hypoxic phase HP and the reoxygenation phase RP in the form of a data curve 46 (see FIG. 4). In a sixth step VI, the hypoxic session index HSI is determined from a difference between the data curve 46 and a predetermined reference curve 48 (see FIG. 4).

FIG. 4 schematically shows a data curve 46 of the index KG, which is recorded in the method 100 of measuring the hypoxic session index 26 against a measurement time MT in the initial phase AP, the hypoxic phase HP and the reoxygenation phase RP. Shown is also a predetermined reference curve 48 with which the data curve 46 is compared. Hypoxic baselines 50a of the reference curve 48, and hypoxic baselines 50b of the data curve 46 are obtained by plotting the mean value of measured values of the index KG against the measurement time MT in the initial phase AP of the method 100. In the reoxygenation phase RP, the reference curve 48 and the data curve 46 rise to the hyperoxic reference value HRW, which is shown here schematically as the same value for the reference curve 48 and the data curve 46. The hypoxic phase HP begins at the start time AZP. In the hypoxic phase, the patient is supplied with a hypoxic gas mixture 14 over a defined hypoxic period until the index KG drops to the hypoxic value 82 that is greater than the defined hypoxic safety value 52, which is plotted as a hypoxic safety value line 52 against the measurement time MT.

A predetermined HCS reference value of the index is smaller than the mean value of the index in the initial phase and is greater than the hypoxic safety value 52. The HCS reference value is shown as the HCS reference line 54 of the hypoxic phase against the measurement time MT. An HCS determination area 56 is defined as an area between the data curve 46 and the HCS reference line 54 in the hypoxic phase HP. An HCS reference area 58 is determined as an area between the reference curve 48 and the HCS reference line 54 in the hypoxic phase, the end of which is represented by a vertical line. The value of the hypoxic cycle score HCS is defined as a measure of the ratio and/or the difference between the HCS determination area 56 and the HCS reference area 58.

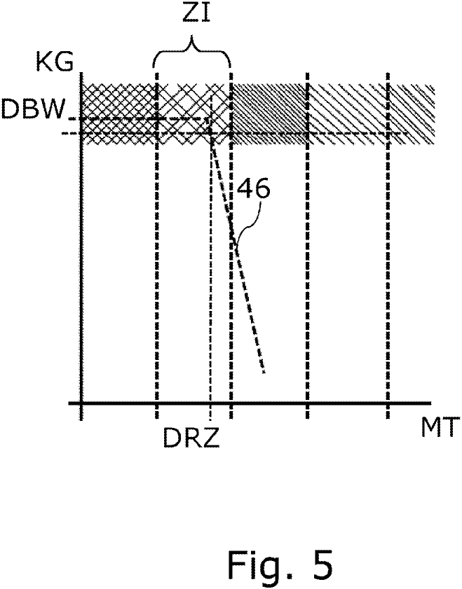

FIG. 5 schematically shows the data curve 46 in the initial phase AP and in the hypoxic phase HP (see FIG. 4) up to a DS reference time DRZ of the measurement time MT, at which DS reference time the index KG has dropped to a defined DS reference value DBW. The data curve 46 is shown schematically as a straight line in the initial phase AP and up to the time at which it starts to drop.

The DS reference value DBW is smaller than the mean value of the index KG in the initial phase AP and is greater than the hypoxic safety value 52 (see FIG. 4). The DS reference value DBW is, in particular, 96% to 98% of the mean value of the index KG from the initial phase AP. The time difference between the DS reference time DRZ and a defined DS reference time interval, in particular a DS reference time interval of 40 seconds to 50 seconds, is recorded as the value of the dynamic score DS, optionally divided by a numerical factor, for example 100. In case of negative time difference values and/or time difference values that are greater than a predetermined value, in particular, more than 184 seconds, the dynamic score DS is preferably set to zero.

Alternatively or additionally, the measurement time in the initial phase AP and the hypoxic phase HP may be divided into time intervals ZI, wherein each time interval ZI is assigned a hypoxic improvement potential, represented by different hatching in the time intervals ZI, such that a position of the DS reference time DRZ in a certain time interval corresponds to a certain hypoxic improvement potential.

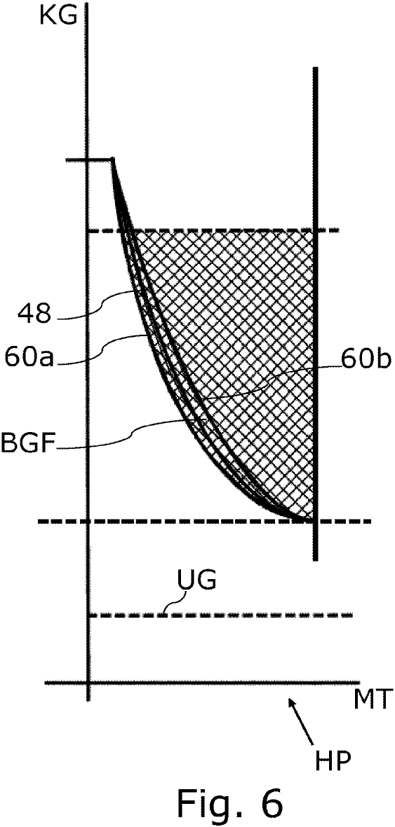

FIG. 6 schematically shows a lower boundary line 60a and an upper boundary line 60b of the index KG in a hypoxic phase HP. The lower boundary line 60a in the hypoxic phase HP has, at the respective measurement time MT, smaller values of the index KG than the reference curve 48, and the upper boundary line 60b has greater values of the index KG than the reference curve 48. Only values of the index KG, which lie in the area BGF defined by the boundary line, are, in particular, connected to one another and used to determine the areas and indices mentioned in the application. The dashed line designated UG represents a lower limit of the index KG in the hypoxic phase HP that can be selected as a hypoxic safety value 52 (see FIG. 4).

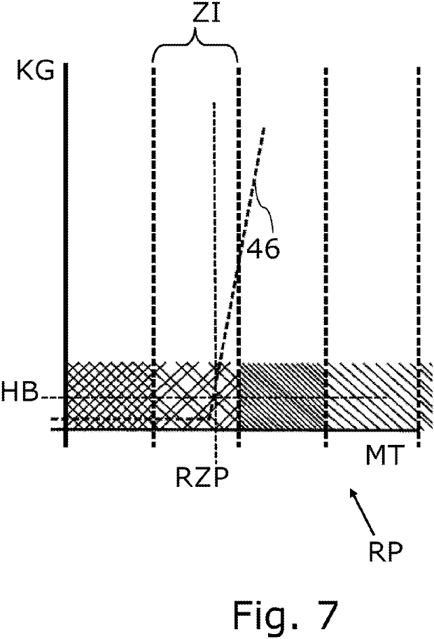

FIG. 7 schematically shows the data curve 46 from the beginning of the reoxygenation phase RP up to an RI reference time RZP of the measurement time MT, at which RI reference time the index KG has increased to a defined hyperoxic reference value HB. The data curve 46 is schematically shown as a straight line from the beginning of the reoxygenation phase RP up until the time when the index KG starts to rise. The hyperoxic reference value HB is, in particular, 2% to 4% of the mean value of the index KG from the initial phase AP.

The time difference between the RI reference time RZP and a defined RI reference time interval, in particular an RI reference time interval of 40 seconds to 50 seconds, is recorded as the value of the reoxygenation impulse score RIS. In case of negative time difference values and/or time difference values that are greater than a predetermined value, in particular, more than 184 seconds, the reoxygenation impulse score RIS is preferably set to zero. Alternatively or additionally, the measurement time MT in the reoxygenation phase RP may be divided into time intervals ZI, wherein each time interval ZI is assigned a reoxygenation improvement potential, represented by different hatching, such that a position of the RI reference time RZP in a certain time interval ZI corresponds to a certain reoxygenation improvement potential.

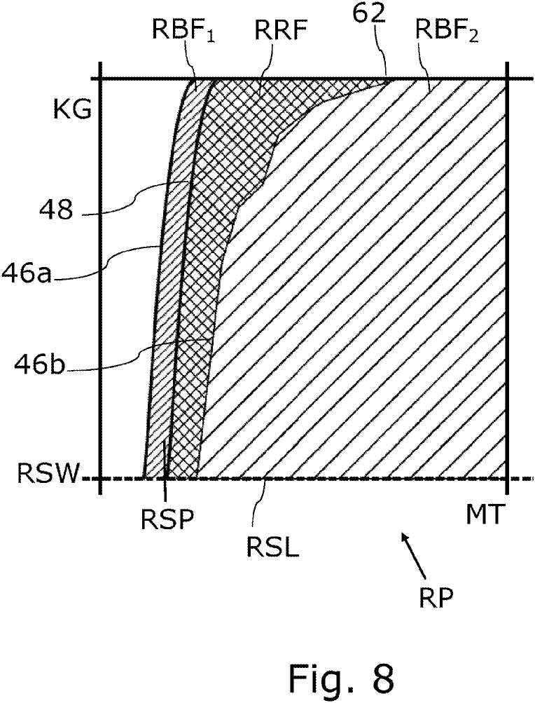

FIG. 8 schematically shows a reference curve 48 as well as a first data curve 46a and a second data curve 46b in the reoxygenation phase RP above an RMS safety value RSW, shown as a dashed, horizontal RMS safety line RSL against the measurement time MT. The RMS safety value is a value of the index KG on the reference curve 48 at an RMS safety time RSP after the beginning of the reoxygenation phase RP.

In this case, the first data curve 46a reaches the RMS safety value RSW at an earlier time than the reference curve 48, and the second data curve 46b reaches the RMS safety value RSW at a later time than the reference curve 48. The horizontal line 62 schematically identifies a maximum value of the index KG, which the reference curve 48, the first data curve 46a and the second data curve 46b adopt in this exemplary embodiment in the reoxygenation phase RP. The cross-hatched area and the roughly hatched area below the reference curve together form the RMS reference area RRF. The reoxygenation max score RMS of the first data curve 46a is defined as the ratio of the total area of finely hatched area, cross-hatched area and roughly hatched area below the first data curve as the RMS determination area RBF₁ and the RMS reference area RRF. The reoxygenation max score RMS of the second data curve is defined as the ratio of the roughly hatched area below the second data curve as the RMS determination area RBF₂ and the RMS reference area RRF.

The respective user reoxygenation potential URP (see FIG. 2) of the first and second data curve is defined as a measure of the difference between a predetermined reference reoxygenation max score, in particular a value between 0.97 and 1, preferably 0.99, and the respective reoxygenation max score RMS of the first and second data curve determined in this way. The respective URP represents a measure of the difference between the first or second data curve 46a, 46b and the reference curve 48 in the reoxygenation phase RP.

FIG. 9 schematically shows the pulse RR of a patient (not shown) over the measurement time MT in the form of a pulse curve 64. The mean value of the pulse RR in the initial phase AP is plotted as a heart rate baseline 66 against the duration of hypoxic training after the initial phase parallel to the time axis of measurement time MT. Here, the heart rate baseline 66 forms a first leg 68a of a relaxation measurement angle RMW. A second leg 68b of the relaxation measurement angle RMW runs through the value of the pulse RR at the start time AZP of a first hypoxic phase HP and through the point 70 of the pulse curve 64 with the lowest value of the pulse RR after the initial phase AP. The heart rate relaxation score HRS is defined as a measure of the relaxation measurement angle RMW. In particular, steps II to V of the method have been repeated several times before the determination of the heart rate relaxation score, wherein the hypoxic phases HP, represented by non-hatched areas, and the reoxygenation phases RP, represented by hatched areas, alternate. Per cycle comprising a hypoxic phase and a subsequent reoxygenation phase, a heart rate dynamic score HDS is determined as the difference between the maximum value of the pulse RR in the cycle, indicated by a horizontal line 72a, and the minimum value of the pulse RR in the cycle, indicated by a horizontal line 72b.

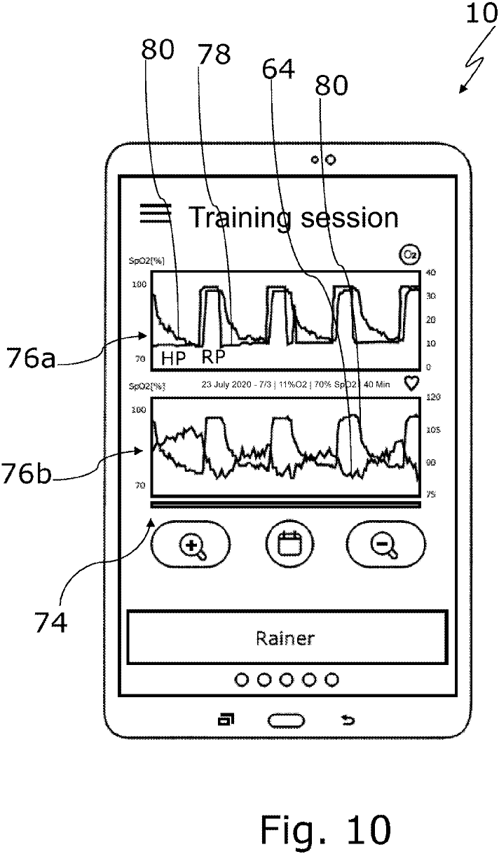

FIG. 10 schematically shows the device 10 with a display 74. The display 74 has a first display panel 76a, where the oxygen O₂ which is supplied to a patient over the measurement time MT is shown as an oxygen curve 78. Shown is, in particular, the varying amount of oxygen in the hypoxic phase HP and in the reoxygenation phase RP. Each hypoxic phase HP is followed by a reoxygenation phase RP. The pertinent values of the index which characterizes the oxygen saturation of the blood of a patient are shown in the form of a partial pressure of oxygen curve 80. The left scale of the first display panel 76a refers to the partial pressure of oxygen SpO2, stated in percent. The right scale refers to the amount of oxygen O₂ in the gas mixture supplied, stated in vol%. The amount of oxygen O₂ in the gas mixture supplied varies, in particular, between 7.5 vol% and 17 vol% in the hypoxic phase HP, and between 20 vol% and 35 vol%, in particular between 20.9 vol% and 32 vol%, preferably between 25 vol% and 30 vol%, in the reoxygenation phase RP. The horizontal axis indicates the measurement time MT during hypoxic training.

The display 74 has a second display panel 76b, where the values of the partial pressure of oxygen SpO2 are plotted as the partial pressure of oxygen curve 80, and the values of the pulse RR of the patient are plotted as a pulse curve 64 against the measurement time MT. The left scale of the second display panel 76b refers to the partial pressure of oxygen SpO2, stated in percent. The right scale refers to the pulse RR, stated in bpm (beats per minute). The horizontal axis indicates the measurement time MT during hypoxic training.

When viewing all figures of the drawing in combination, the invention relates to a method 100 of measuring a hypoxic session index HSI as a measure of the response of a patient to hypoxic training, with measurement of an index KG that characterizes the oxygen content in the patient’s blood. The index KG is, in particular, the oxygen saturation and/or the partial pressure of oxygen SpO2. The patient is first supplied with a normoxic gas mixture 16 in an initial phase AP. Subsequently, in a hypoxic phase HP, the patient is supplied with a hypoxic gas mixture 14 over a defined hypoxic period. Subsequently, the patient is supplied with a normoxic or hyperoxic gas mixture 16 in a reoxygenation phase RP, in particular over a period of 1 minute to 10 minutes. The reoxygenation phase RP extends from the time from which the patient is supplied with the normoxic or hyperoxic gas mixture 16, over a defined hyperoxic period, wherein the index KG attains a hyperoxic reference value HRW of the index KG, and over a subsequent predetermined concluding period. The hypoxic session index HSI is determined from the differences between a data curve 46, 46a, 46b having the measurements of the index plotted against the respective measurement time MT of the index KG and a predetermined reference curve 48.

List of reference signs:

| | |
|---|---|
| 10 | Apparatus |
| 12 | Mask |
| 14 | Hypoxic gas mixture |
| 16 | Normoxic and/or hyperoxic gas mixture |
| 18 | Controller |
| 20 | Finger clip |
| 22 | Sensor |
| 24 | Mobile application |
| 26 | Indication value |
| 45 | Hear rate dynamic score |
| 46, 46a, b | Data curve |
| 48 | Reference curve |
| 50a, b | Hypoxic baselines |
| 52 | Hypoxic safety value line |
| 54 | HCS reference line |
| 56 | HCS determination area |
| 58 | HCS reference area |
| 60a, b | Lower, upper boundary line |
| 62 | Maximum value of the reference curve and of the first, second data curve in the reoxygenation phase |
| 64 | Pulse curve |
| 66 | Hear rate baseline |
| 68a, b | Leg |
| 70 | Point of pulse curve 64 with the lowest value of the pulse |
| 72a, b | Maximum, minimum value of the pulse |
| 74 | Display |
| 76a, b | Display panels |
| 78 | Oxygen curve |
| 80 | Partial pressure of oxygen curve |
| 82 | Hypoxic value |
| 100 | Method |
| AP | Initial phase |
| AZP | Start time of the first hypoxic phase |
| BGF | Area defined by the boundary line |
| BP | Baseline potential |
| DBW | DS reference value |
| DRZ | DS reference time |
| DS | Dynamic score |
| HB | Hyperoxic reference value |
| HCS | Hypoxic cycle score |
| HDS | Hear rate dynamic score |
| HP | Hypoxic phase |
| HRS | Heart rate relaxation score |
| HRW | Hyperoxic reference value |
| HSI | Hypoxic session index |
| KG | Index |
| MT | Measurement time |
| ORS | Oxygen recovery score |
| RBF | RMS determination area |
| RIS | Reoxygenation impulse score |
| RMS | Reoxygenation max score |
| RMW | Relaxation measurement angle |
| RP | Reoxygenation phase |
| RR | Pulse |
| RRF | RMS reference area |
| RSL | RMS safety line |
| RSP | RMS safety time |
| RSW | RMS safety value |
| RZP | RI reference time |
| SBL | Hypoxic baseline (SPO2 baseline) |
| UG | Lower limit of the index in the hypoxic phase |
| URP | User reoxygenation potential |
| ZI | Time intervals |

What is claimed is:

1. A method of measuring a hypoxic session index (HSI) as a measure of the response of a patient to hypoxic training, with measurement of an index (KG) that characterizes the oxygen saturation in the patient’s blood, having the steps:
I) supplying the patient with a normoxic gas mixture in an initial phase (AP) over a defined first period;
II) determining the mean value of the index (KG) in the initial phase;
III) supplying the patient with a hypoxic gas mixture in a hypoxic phase (HP) over a defined hypoxic period;
IV) supplying the patient with the normoxic or hyperoxic gas mixture in a reoxygenation phase (RP) over a defined hyperoxic period and in a subsequent predetermined concluding period;
V) plotting the index (KG) against time in the initial phase (AP), the hypoxic phase (HP) and the reoxygenation phase (RP) in the form of a data curve;
VI) determining the hypoxic session index from a difference between the data curve and a predetermined reference curve (48);
wherein the reference curve is a data curve which is determined for a reference person after steps I to V, or a data curve which is calculated by averaging data curves of a plurality of reference persons, wherein the data curves are determined after steps I to V for the reference persons.

2. The method according to claim 1, including setting a hypoxic cycle score (HCS) in the hypoxic phase (HP) for determining the hypoxic session index (HSI) by the following steps:
VII) setting an HCS reference value of the index (KG) that is smaller than the mean value of the index (KG) in the initial phase (AP) and is greater than a hypoxic safety value;
VIII) plotting the HCS reference value against time in the hypoxic phase (HP) as an HCS reference line of the hypoxic phase (HP);
IX) determining an HCS determination area as an area between the data curve and the HCS reference line in the hypoxic phase (HP);
X) determining an HCS reference area as an area between the reference curve and the HCS reference line in the hypoxic phase (HP);
XI) determining an HCS area difference between the HCS reference area and the HCS determination area and/or an HCS area ratio as the ratio of the HCS determination area and the HCS reference area;
XII) determining the value of the hypoxic cycle score (HCS) as a measure of the HCS area ratio and/or the HCS area difference.

3. The method according to claim 1, including setting a reoxygenation max score (RMS) in the reoxygenation phase (RP) for determining the hypoxic session index (HSI) by the following steps:
XIII) setting an RMS safety time (RSP) after the start of the reoxygenation phase (RP);
XIV) determining the RMS safety value (RSW) of the index (KG) associated with the RMS safety time (RSP) on the data curve in the reoxygenation phase (RP);
XV) plotting the RMS safety value (RSW) against time (MT) in the reoxygenation phase (RP) as an RMS safety line (RSL) of the reoxygenation phase (RP);
XVI. determining an RMS determination area (RBF₁, RBF₂) as an area between the data curve and the RMS safety line (RSL) in the reoxygenation phase (RP);
XVII) determining an RMS reference area (RRF) as an area between the reference curve and the RMS safety line (RSL) in the reoxygenation phase (RP);
XVIII) determining an RMS area difference between the RMS reference area (RRF) and the RMS determination area (RBF₁, RBF₂) and/or an RMS area ratio as the ratio of the RMS determination area (RBF₁, RBF₂) and the RMS reference area (RRF);
XIX) determining the reoxygenation max score (RMS) as a measure of the RMS area ratio and/or the RMS area difference.

4. The method according to claim 1, characterized by determining a user reoxygenation potential (URP) as a measure of the difference between a predetermined reference reoxygenation max score (RMS) and the reoxygenation max score (RMS) determined in step XIX.

5. The method according to claim 3, including setting a dynamic score (DS) in the hypoxic phase (HP) for determining the hypoxic session index (HSI), having the steps:
XX) determining a DS reference time (DRZ) at which the index (KG) in the hypoxic phase (HP) has dropped to a defined DS reference value (DBW), wherein the DS reference value (DBW) is smaller than the mean value of the index (KG) in the initial phase (AP) and greater than the hypoxic safety value;
XXI) determining the dynamic score (DS) as a measure of the time difference between the DS reference time (DRZ) and a defined DS reference time interval.

6. The method according to claim 1, including setting a reoxygenation impulse score (RIS) in the reoxygenation phase (RP) for determining the hypoxic session index (HSI), having the steps:
XXII) determining an RI reference time (RZP) to which the index (KG) in the reoxygenation phase (RP) has increased to a defined hyperoxic reference value (HB);
XXIII) determining the reoxygenation impulse score (RIS) as a measure of the time difference between the RI reference time (RZP) and a defined RI reference time interval.

7. The method according to claim 1, including setting an oxygen recovery score (ORS) in the reoxygenation phase (RP) for determining the hypoxic session index (HSI) by the reoxygenation impulse score (RIS) and the reoxygenation max score (RMS), by averaging.

8. The method according to claim 1, including determining a baseline potential (BP) as a measure of the difference between a predetermined ideal value of the index (KG) of the oxygen saturation and the mean value of the index (KG) of the oxygen saturation from the initial phase (AP).

9. The method according to claim 1, including setting a lower boundary line and an upper boundary line, wherein the lower boundary line in the hypoxic phase (HP) shows smaller values of the index KG than the reference curve (48), and the upper boundary line shows greater values of the index (KG) than the reference curve at the respective same measurement times, wherein the boundary lines are determined from measurements of the index (KG) in one or more subjects, wherein only values of the index (KG) are considered for determining the hypoxic session index (HSI), which values are smaller than the value of the index (KG) in the upper boundary line and greater than the value of the index (KG) in the lower boundary line at the time of measurement of the respective value of the index (KG).

10. The method according to claim 1, including determining the hypoxic session index (HSI) by changing a pulse curve of the patient during steps III to V.

11. The method according to claim 10, including determining a heart rate relaxation score (HRS) by plotting a pulse curve against time during hypoxic training, having the following steps:
XXIV) determining the mean value of the pulse (RR) in the initial phase (AP);
XXV) plotting the mean of the pulse (RR) against the duration of the hypoxic training after the initial phase (AP) as a heart rate baseline parallel to the time axis, wherein the heart rate baseline forms a first leg of a relaxation measurement angle (RMW);
XXVI) plotting a second leg of the relaxation measurement angle (RMW), wherein the second leg runs through the pulse (RR) at the start time (AZP) of the hypoxic phase (HP) and through the point of the pulse curve having the lowest value of the pulse (RR) after the initial phase (AP);
XXVII. Determining the heart rate relaxation score (HRS) as a measure of the relaxation measurement angle (RMW).

12. The method according to claim 1, including one or more repetitions of steps III to V before determining the hypoxic session index (HSI) according to step VI.
